(19) United States
(12) Patent Application Publication
(10) Pub. No.: US 2026/0354989 A1
(43) Pub. Date: Nov. 12, 2026

(54) EXTERNAL BREAST PROSTHESES

(71) Applicant: Board of Regents, The University of Texas System, Austin, TX (US)

(72) Inventor: Yunlan ZHANG, Austin, TX (US)

(21) Appl. No.: 19/670,815

(22) Filed: May 7, 2026

Related U.S. Application Data
(60) Provisional application No. 63/801,658, filed on May 7, 2025.

(51) Int. Cl. A61F 2/52; A61F 2/50; A61F 2/78

(52) U.S. Cl. CPC A61F 2/52; A61F 2/78; A61F 2002/505

(57) ABSTRACT

Embodiments of the present subject matter generally relate to prosthetic devices and methods of designing and manufacturing prosthetic devices. Specifically, the devices and methods disclosed herein incorporate a plurality of architected structures and computational design frameworks to achieve target physical and functional properties of an external prosthesis. The external prosthesis includes an outer body comprising a shape configured to approximate a breast and an internal structure disposed within the outer body, the internal structure comprising a plurality of architected structures having an internal geometry, the internal geometry comprising a plurality of unit cells arranged to define a three-dimensional topology. The plurality of unit cells include a defined density, a mechanical compliance, and a thermal conductivity.

### CROSS-REFERENCE TO RELATED APPLICATIONS

This application claims the benefit of U.S. Provisional Application No. 63/801,658, filed on May 7, 2025, which is incorporated by reference herein in its entirety.

### BACKGROUND

### Field

Embodiments of the present subject matter generally relate to prosthetic devices and methods of designing and manufacturing prosthetic devices. Specifically, the devices and methods disclosed herein incorporate a plurality of architected structures and computational design frameworks to achieve target physical and functional properties of an external prosthesis for both short-term and long-term usage.

### DESCRIPTION OF THE RELATED ART

Breast cancer remains a highly prevalent condition worldwide, with mastectomies frequently performed as part of treatment or risk-reduction strategies. Post-surgery, breast prostheses are essential for restoring appearance, balance, and confidence. External breast prostheses are widely used as a non-surgical option to restore body symmetry, improve postural balance, and patient well-being. However, conventional external prostheses often fall short in addressing key challenges such as comfort, weight, heat management, and personalization. In particular, conventional prostheses are often formed as homogeneous structures that may not accurately replicate the physical characteristics of natural breast tissue.

Conventional prostheses may exhibit densities that differ from natural breast tissue, which can contribute to asymmetric loading and discomfort, particularly in unilateral applications. In addition, natural breast tissue exhibits heterogeneous mechanical properties, whereas conventional prostheses typically provide a uniform mechanical response that may not accurately mimic native tissue behavior. Thermal performance also presents a challenge, as prostheses with relatively low thermal conductivity can result in heat buildup at the interface between the prosthesis and the wearer.

Accordingly, there remains a need for improved prosthetic devices and associated design and manufacturing methods that enable patient-specific optimization of weight balance, thermal performance, and mechanical compliance.

### SUMMARY

In a first embodiments, an external prosthesis is disclosed. The external prosthesis includes an outer body comprising a shape configured to approximate a breast and an internal structure disposed within the outer body, the internal structure comprising a plurality of architected structures having an internal geometry, the internal geometry comprising a plurality of unit cells arranged to define a three-dimensional topology. The plurality of unit cells includes a defined density, a mechanical compliance, and a thermal conductivity.

In another embodiment, an external breast prosthesis (EBP) is disclosed. The EBP including a first region including a first architected structure, wherein the first architected structure is configured to simulate an adipose tissue; and a second region including a second architected structure, wherein the second architected structure is configured to simulate a glandular tissue, wherein the first region and the second region are integrated to form a unitary structure.

In another embodiments, a method of forming an external breast prosthesis (EBP) is disclosed. The method includes collecting a plurality of anthropometric data from a patient, determining a plurality of design objectives based on at least the anthropometric data, translating the plurality of anthropometric data and the plurality of design objectives into a plurality of design parameters associated with a plurality of architected structures, the design parameters comprising relative density, unit cell geometry (wavelength, cell wall thickness, curvatures, porosity etc.), topology, or combinations thereof, generating a digital model of the EBP based on the plurality of design parameters, the digital model including an internal structure comprising the plurality of architected structures, and forming the EBP based on the digital model using an additive manufacturing process.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIGS. 1A and 1B illustrate a schematic representation of a patient and an external prosthesis, according to certain embodiments.

FIG. 2 illustrates a sagittal cross-sectional diagram of a human breast, according to certain embodiments.

FIGS. 3A, 3B, 3C, and 3D illustrate three-dimensional (3D) models and 3D-printed samples, according to certain embodiments.

FIGS. 4A, 4B, and 4C illustrate schematic representations of variations in sample material, according to certain embodiments.

FIG. 5 illustrates the effect of wavelength on the compressive modulus and thermal conductivity, according to certain embodiments.

FIG. 6 illustrates a stress-strain curve for sample prostheses and simulated prostheses, according to certain embodiments.

FIGS. 7A and 7B illustrate a temperature distribution of gyroid structures during a steady state, according to certain embodiments.

FIG. 8 shows the finite element model for the thermal conductivity analysis, according to certain embodiments.

FIG. 9 shows the finite element models of the compressive process for gyroid structures, according to certain embodiments.

FIG. 10 illustrates a patient timeline related to production of the external prosthesis, according to certain embodiments.

FIG. 11 illustrates a flow diagram of a method of fabricating the external prosthesis, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present subject matter generally relate to prosthetic devices and methods of designing and manufacturing prosthetic devices. Specifically, the devices and methods disclosed herein incorporate a plurality of architected structures and computational design frameworks to achieve target physical and functional properties of an external prosthesis.

Breast cancer is a highly prevalent disease worldwide, with over two million diagnoses annually. Treatment strategies vary based on cancer types, stages, and individual patient factors, and encompass endocrine therapy, chemotherapy, radiation, and surgery, often utilized in combination. Chemotherapy is frequently employed to shrink tumors, particularly in more aggressive or advanced cases, making surgical interventions more effective by reducing the size of the cancerous tissue prior to removal. While a lumpectomy involves removing the tumor while preserving most of the breast tissue, a mastectomy entails the removal of the entire breast. Mastectomy is particularly important in cases where the cancer is more advanced, cannot be adequately addressed by lumpectomy, or when the patient has a genetic predisposition to breast cancer, such as breast cancer gene (BRCA) mutations, necessitating a more aggressive approach. Mastectomy rates vary across regions and over time, with about 30% to about 60% of breast cancer patients undergoing the procedure, highlighting its significant role in breast cancer management. Additionally, a mastectomy is often performed prophylactically in individuals with a high genetic risk to prevent the development of breast cancer. Accordingly, post-mastectomy rehabilitation is an important component of breast cancer care.

Despite mastectomy being an effective treatment and preventive measure, it has profound implications for a patient's psychological well-being, body image, balance, posture, and overall comfort. Post-mastectomy patients have several options to address changes in their appearance, including reconstructive surgery with a breast implant, external breast prostheses (EBPs), or choosing to “go flat,” meaning they abstain from reconstruction and prosthesis use. For many, particularly those without adequate health insurance or access to reliable healthcare, EBPs are the preferred solution. Typically made from silicone, the EBPs aim to replicate the appearance and feel of natural breast tissue, providing benefits such as improved posture and self-image. However, EBPs are not without drawbacks, as the weight, heat accumulation, and limited personalization of the conventional EBPs are major sources of user dissatisfaction. For instance, heavy EBPs can cause shoulder pain and fatigue, while silicone-based prostheses often cause discomfort due to heat buildup, especially during warmer months. Density mismatches between EBPs and natural breast tissue can contribute to asymmetric loading, particularly in unilateral applications, and the generally homogenous construction of EBPs does not replicate the heterogenous mechanical properties of natural breast tissue, which includes both adipose and glandular components.

Despite ongoing efforts to enhance comfort and functionality, conventional EBPs remain constrained by the complexity and high cost of traditional manufacturing methods. Some commercial designs, for instance, incorporate air-inflatable systems to better conform to the chest wall, but these solutions are often expensive and dependent on bulky external components. In response to the referenced limitations, additive manufacturing (AM) has emerged as a transformative technology, offering control over structure, material distribution, and personalization. In recent years, AM has been widely explored across biomedical domains such as intervertebral disc repair, soft tissue scaffolding, and reconstructive surgery. For example, multiphasic 3D-printed scaffolds that combine polymeric backbones with hydrogels have been developed to replicate the mechanical gradients of intervertebral discs, while poly (ester amide)-modified scaffolds have demonstrated enhanced stiffness and bioactivity for soft tissue regeneration. In the context of breast reconstruction, AM and tissue engineering have enabled the development of patient-specific anatomical models and integrated soft-tissue implants. Recently, AM has been explored for prosthetic applications to enable patient-specific geometries derived from imaging or scanning technologies; however, many approaches primarily focus on replicating external shape rather than engineering internal structure to achieve desired functional performance.

Building on the broader applications of AM in biomedical engineering, researchers and companies have also begun to explore the potential in the development of customized EBPs. Several academic and commercial initiatives have emerged in recent years, applying digital design and 3D printing technologies to create more individualized and functionally adaptive breast prosthetic solutions. For example, there is a strong focus on advancing 3D printing technology and expanding 3D printing technology to include other soft tissue implants. Further, scanning technology is used to generate 3D files for producing silicone EBPs or multi-layered EBPs. The mentioned 3D printing technologies focus on improved individual fit and user experience, however the technologies neglect the need to mimic the biomechanical and thermal properties of natural breast tissue. As a result, challenges related to comfort, weight distribution, and heat retention persist in currently available EBPs. In addition, there remains a lack of systematic design frameworks that translate patient-specific anatomical data and user-defined performance preferences into EBP designs optimized across multiple performance characteristics, including weight balance, thermal conductivity, and mechanical compliance.

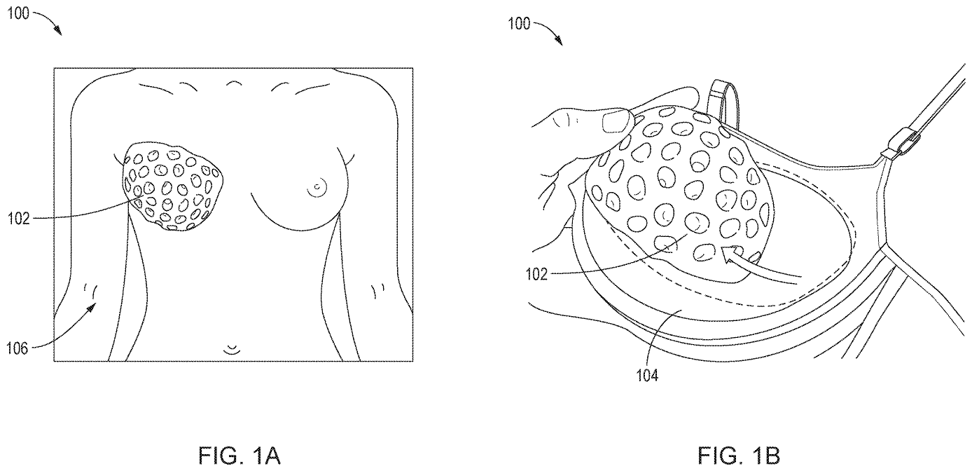

As disclosed herein, the subject matter integrates digital design, numerical simulation, and AM to engineer an external breast prosthesis (EBP) 102 (e.g., as shown in FIGS. 1A and 1B). Unlike conventional solutions that focus solely on surface customization, embodiments described herein employ an architected material system (e.g. a plurality of architected structures) enabling programmable control over both mechanical and thermal performance at the microstructural level. As used herein, the term “an architected structure” or “a plurality of architected structures” refers to structures having engineered internal geometries, including triply periodic minimal surface (TPMS)-based, spinodal, stochastic cellular, or lattice-based configurations, that are configured to achieve one or more target physical properties, such as density, mechanical compliance, or thermal performance.

In one or more embodiments, the plurality of architected structures include a plurality of unit cells arranged to define a three-dimensional topology, wherein geometric parameters of the unit cells are selected to provide a defined density, mechanical compliance, and thermal conductivity. This strategy enables the creation of the EBP 102 that more accurately replicate the physical properties of natural breast tissue, addressing post-mastectomy concerns such as balance, pressure distribution, and thermal comfort. By tuning the wavelength and wall thickness of the plurality of architected structures, embodiments of the disclosure enable precise modulation of stiffness, density, and thermal conductivity to establish data-driven design guidelines. The plurality of architected structures are fabricated using soft, deformable polymer materials that are operable to mimic the compressive behavior and tactile response of biological tissue. For example, a material such as TissueMatrix™ (MED310C) is used to form the plurality of architected structures. Additionally, three-dimensional imaging, magnetic resonance imaging (MRI), computed tomography (CT), or combinations thereof scans can be utilized to design prosthetics precisely tailored to each patient's anatomy and functional needs. This strategy has the potential to transform the EBP 102 by providing enhanced comfort, function, and access to individualized post-mastectomy care.

A design approach for the EBP 102 that leverages architected material systems to optimize both comfort and functionality, closely replicating the density, thermal conductivity, and mechanical behavior of natural breast tissue is disclosed herein. The plurality of architected structures includes a plurality of unit cells. By tailoring a geometry of the plurality of unit cells, it is possible to achieve a tissue-like density that ensures a balanced weight and natural feel. Additionally, optimizing thermal conductivity prevents heat buildup, maintaining a cool and comfortable interface. Furthermore, the stiffness of the material can be closely matched to that of breast tissue, enhancing biomechanical compatibility and overall functionality. Leveraging advancements in AM, this approach utilizes additively manufactured, biocompatible materials, offering a cost-effective alternative to traditional prostheses and reducing financial barriers for patients.

FIG. 1A illustrates an example prosthetic system 100 including the EBP 102 positioned on a patient 106. In one or more embodiments, the EBP 102 is configured to be worn externally on a chest wall of the patient 106, for example following a mastectomy or other medical procedure. The EBP 102 may be shaped and dimensioned to approximate the size, contour, and mass distribution of natural breast tissue, thereby restoring symmetry and improving comfort and confidence for the patient 106. As shown, the EBP 102 may be positioned directly against the patient 106 or in combination with a support structure 104, as described further below.

FIG. 1B illustrates the prosthetic system 100 in which the EBP 102 is positioned within an EBP support structure 104. In one or more embodiments, the EBP support structure 104 may include a garment, such as a brassiere, pocketed garment, or other wearable structure configured to receive and retain the EBP 102. The EBP support 104 may be configured to maintain positioning of the EBP 102 relative to the patient 106 during use, while allowing for insertion, removal, and adjustment of the EBP 102. In one or more embodiments, the EBP 102 may be removably coupled to the EBP support structure 104, enabling interchangeability of prostheses having different properties, such as weight, stiffness, or thermal characteristics.

As disclosed herein, the EBP 102 includes an outer body including a shape configured to approximate a breast and an internal structure disposed within the outer body. The internal structure includes the plurality of architected structures. The plurality of architected structures include the plurality of unit cells arranged to define a three-dimensional topology. The unit cells include a defined density, a mechanical compliance, and a thermal conductivity, each of which is controlled by one or more geometric parameters of the unit cells, including relative density, topology, and wavelength. In one or more embodiments, the EBP 102 includes a first region and a second region. The first region includes a first architected structure configured to simulate the adipose tissue. The second region includes a second architected structure configured to simulate the glandular tissue. The first region and the second region are integrated to form a unitary structure.

### Tissue Composition of the Breast

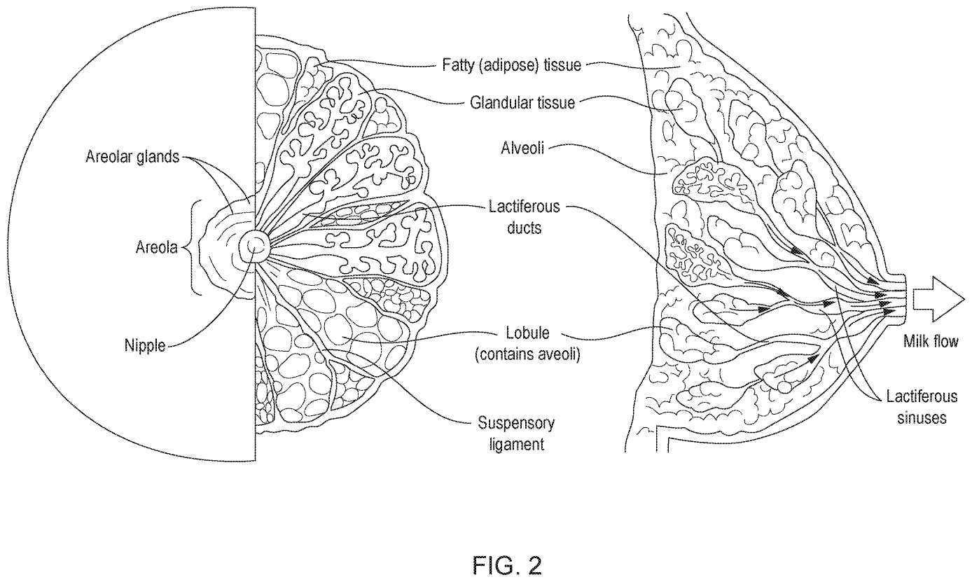

The human breast primarily consists of two types of tissue: adipose tissue (fat), which provides structural support and energy storage, and glandular tissue (milk-producing), which is specialized for lactation and milk production, as shown in FIG. 2. Glandular tissue is denser than adipose tissue and is formed in lobes that extend radially from the nipple; adipose tissue fills the rest of the volume encompassed by the skin. Glandular tissue has a density of approximately about 1.05 g/cm3 to about 1.08 g/cm3 and adipose tissue has a density of about 0.4 g/cm3 to about 0.6 g/cm³. The mechanical properties of these tissues are commonly characterized by their compressive modulus; however, the measured values can vary substantially due to multiple factors. These include the degree of pre-strain applied during compression tests, the experimental conditions (e.g., whether measurements are conducted ex vivo or in vivo), and the source or origin of the tissue sample. Furthermore, there is variation in breast volume and density of glandular tissue to the overall volume. Factors like age, weight, body max index (BMI), and hormonal patterns can influence both breast volume and density. Based on the literature review, the median breast has a volume of approximately 494 ml, with about 24% of the volume consisting of fibroglandular tissue.

To replicate the density and mechanical properties of breast tissue in an external prosthesis, it is desirable to account for the heterogeneous composition of adipose regions (e.g., the first region) and glandular regions (e.g., the second region) and their respective spatial distributions. For example, the plurality of architected structures includes a variety of properties in different regions to represent the heterogenous composition of breast tissue. The plurality of unit cells within the plurality of architected structures are operable to be modified to conform to the composition of different regions. The plurality of architected structures are operable to be integrated together. In one or more embodiments, as disclosed herein, glandular tissue is simulated by forming one or more relatively dense regions, while adipose tissue is simulated using the plurality of architected structures having tailored internal geometries. The plurality of architected structures may include triply periodic minimal surface (TPMS)-based, spinodal, lattice-based, stochastic cellular structures configured to provide a target density, mechanical compliance, and thermal performance. In this manner, the relative proportions and distributions of adipose and glandular analog regions are selected to approximate patient-specific tissue composition and associated physical properties.

In one or more embodiments, the internal structure includes a spatially varying or graded architected structure in which one or more geometric parameters, such as relative density, wavelength, unit cell size, or topology, vary continuously or semi-continuously across the volume of the prosthesis. The spatial variation is used to create gradual transitions between regions configured to simulate adipose tissue and regions configured to simulate glandular tissue, thereby more closely approximating the heterogeneous and continuously varying properties of natural breast tissue. In one or more embodiments, the graded structure is defined using a continuous parameter field derived from patient-specific anatomical data.

### Architected Composite Structure

The plurality of architected structures are formed from a class of materials engineered to exhibit desired properties, such as high surface-to-volume ratios, controlled stiffness, density, and thermal performance, through the precise control of their geometry and topology. Leveraging advancements in AM of soft materials, the plurality of architected structures offer solutions for creating comfortable prosthetics that replicate the softness, flexibility, and weight distribution of natural tissues while efficiently dissipating heat. This approach enables the design of prosthetic devices that can enhance comfort and improve patient functionality and wearability. In one or more embodiments, the geometry and topology of the plurality of architected structures is selected based on one or more design objectives, such as target density, mechanical compliance, or thermal conductivity. In one or more embodiments, the geometry and topology of the plurality of architected structures are selected based on one or more design objectives, such as target density, mechanical compliance, or thermal conductivity.

In one or more embodiments, the material for the plurality of architected structures include, architected materials, lattice structures, triply periodic minimal surface (TPMS) materials, closed- or open-cells foam materials, cellular materials, porous solids, or combinations thereof. For example, TPMS-based architected materials characterized by continuous, non-self-intersecting surfaces that extend periodically in three dimensions, offer versatility.

In one or more embodiments, the plurality of architected structures include gyroid structures, diamond structures, Schwarz primitive structures, lattice-based structures, stochastic cellular structures, or combinations thereof. TPMS architected materials include a high strength-to-weight ratio, low density, and complex geometries. Further, TPMS materials can be customized for specific applications, allowing for the fine-tuning of mechanical and thermal properties. One TPMS geometry, the gyroid structure, is particularly suited for creating artificial tissues and prosthetics. The gyroid structure includes a highly interconnected pore network, which mimics the natural behavior of soft tissues, providing optimized mechanical performance, isotropic strength, and heat dissipation. The disclosed properties make gyroid-based structures ideal for EBPs, ensuring a more comfortable, breathable, and anatomically accurate solution that addresses both physical and emotional needs. Further, it is contemplated that the prostheses disclosed herein may be used for other portions of the body based on need due to the architected materials' customizable nature, including but not limited to limb prosthetics, facial prosthetics, soft tissue supports, or other anatomically conforming devices.

In one or more embodiments, the plurality of architected structures that are gyroid-based is combined with a relatively solid or less porous region to form a composite structure that mimics breast tissue. For example, the EBP 102 includes the gyroid-based architected structure that simulates the adipose tissue and a more solid region that simulates the glandular tissue. In one or more embodiments, the gyroid-based architected structure and the solid region are formed from a same material with different parameters. In one or more embodiments, the gyroid-based architected structure and the solid region are formed from different materials, different parameters, or combinations thereof. The gyroid-based architected structure and the solid region are integrated, bonded, or otherwise coupled to form a unitary structure.

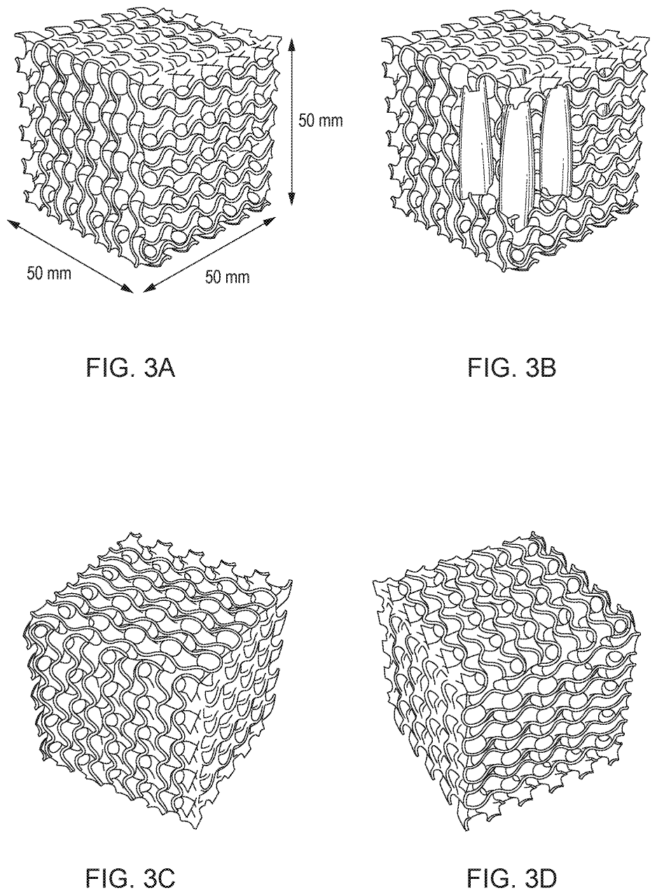

FIGS. 3A, 3B, 3C, and 3D illustrate example embodiments of the plurality of architected structures and corresponding fabricated samples. In particular, FIGS. 3A and 3B illustrate example 3D digital models of the plurality of architected structures having varying geometric configurations, while FIGS. 3C and 3D illustrate corresponding additively manufactured samples. As shown, the internal geometry of the plurality of architected structures is varied to achieve different structural and functional characteristics. In one or more embodiments, the variations in geometry include changes in wavelength, relative density, topology, or combinations thereof.

In one or more embodiments, the digital models illustrated in FIGS. 3A and 3B are generated as part of a parametric design process in which one or more geometric parameters are systematically varied to explore a range of candidate structures. The corresponding fabricated samples illustrated in FIGS. 3C and 3D demonstrate translation of the digital models into physical structures using AM techniques. In one or more embodiments, the different configurations illustrated in FIGS. 3A-3D are used for comparative evaluation of mechanical, thermal, or density-related properties, thereby informing selection of design parameters for a target prosthesis.

For example, in one or more embodiments, a soft, deformable polymer material is used to fabricate one or more portions of the EBP 102. The materials include elastomeric or polymer-based materials configured to approximate the mechanical response of biological tissue. In certain embodiments, a material such as TissueMatrix™ (MED310C) is used.

In one or more embodiments, the physical and functional properties of the EBP 102 are tailored by selecting and adjusting one or more geometric parameters for a unit cell of the plurality of unit cells within the plurality of architected structures. The disclosed parameters include, for example, relative density, characteristic length scale (e.g., wavelength), and structural topology. By varying these parameters, the resulting structure is configured to achieve target properties, including density, mechanical compliance, and thermal conductivity, that approximate those of natural breast tissue. Accordingly, the following sections describe example approaches for relating geometric parameters of the plurality of architected structures to corresponding physical properties, thereby enabling customization of the EBP 102 based on desired performance characteristics and, in some cases, patient-specific inputs.

### Relative Density

The properties of the plurality of architected structures (e.g., the gyroid structures) can be precisely controlled through their geometry to match both the density and mechanical characteristics of adipose tissue. In one or more embodiments, the geometric control enables customization of the EBP 102 properties to approximate patient-specific tissue characteristics and performance objectives. A trigonometric function mathematically describes the gyroid surface itself, as shown in Formula (I).

$$sin ( ( \frac{2 π}{L} ) x ) cos ( ( \frac{2 π}{L} ) y ) + sin ( ( \frac{2 π}{L} ) y ) cos ( ( \frac{2 π}{L} ) z ) + sin ( ( \frac{2 π}{L} ) z ) cos ( ( \frac{2 π}{L} ) x ) - C = 0 \qquad ( I )$$

In Formula (I), L represents the wavelength, which determines the unit cell size, and C controls the volume surrounded by the gyroid surface, where C=0 for purposes of analysis. In Formula (I), x, y, and z represent spatial coordinates in three-dimensional space defining the periodic structure of the gyroid surface. The geometric parameters determine the relative density of the selected architected structure, defined as the ratio between the density of the architected structure (ρ) and the density of the solid base material (ρₛ). For example, the architected structure selected is a gyroid structure. However, as disclosed above, other structures are contemplated for use to form the EBP 102. If a different architected structure is used to form the EBP 102, a different trigonometric function is used to mathematically describe the surface of the architected structure. Relative density is a critical factor that affects the density, mechanical performance, and heat dissipation capabilities. Thus, controlling relative density enables tuning of multiple functional properties of the architected structure. In one or more embodiments, relative density is selected based on one or more design objectives, such as target weight, balance, or comfort, and is varied to achieve desired performance characteristics of the prosthesis. Based on the rule of mixtures, target density of the overall breast tissues (ρₜ) can be approximated using Formula (II), where V_{g} and Vₐ represent the volume of the glandular tissue and the adipose tissue.

$${ρ}_{t} = \frac{{V}_{g} {ρ}_{g} + {V}_{a} {ρ}_{a}}{{V}_{g} + {V}_{a}} \qquad ( II )$$

In one or more embodiments, the EBP 102 is configured to mimic a target density range associated with breast tissue, including glandular tissue, adipose tissue, or combinations thereof. The target density may be selected based on reported density ranges of glandular tissue and adipose tissue, patient-specific anatomical data, anthropometric measurements, user preferences, or combinations thereof. In one or more embodiments, the densities of glandular tissue (pg) and adipose tissue (ρₐ) range from about 1.05 to about 1.08 g/cm³ and about 0.4 to about 0.6 g/cm³, respectively. Hence, the target density of the overall breast tissue is estimated to range between 0.55 and 0.71 g/cm³. In one or more embodiments, the referenced target density values are adjusted based on patient-specific anatomical data, anthropometric measurements, or user preferences, thereby enabling customization of the EBP 102 to more closely match individual tissue characteristics.

The density of the composite structure is also estimated following the rule of mixtures, as described in Formula (III), where pa is the density of the designed gyroid structure with glandular, which should match the target density. ρₛ and ρ represent the density of the printing material and gyroid. In one or more embodiments, the density of the base material is selected based on manufacturing or material considerations. For example, a soft polymer material having a density of approximately 1.2 g/cm³ is used as the base material. Based on the material selection, the relative density of the architected structure is determined, and in certain embodiments is in a range of about 0.28 to about 0.46.

$${ρ}_{d} = \frac{{V}_{g} {ρ}_{s} + {V}_{a} ρ}{{V}_{g} + {V}_{a}} \qquad ( III )$$

### Mechanical Behavior

The mechanical behavior of the plurality of architected structures, including gyroid structures, can be categorized into four distinct stages under the compressive loading condition. In the first, the elastic deformation stage, stress and strain maintain a linear relationship, where the slope of the stress-strain curve represents the compressive modulus. The second stage, the elastic-plastic deformation stage, is marked by a departure from linearity in the stress-strain response. After reaching ultimate strength, the structure enters the plateau stage, where stress remains relatively constant despite increasing strain. Finally, in the failure stage, stress rises sharply as strain continues to increase. Given that prosthetic applications typically operate under relatively small deformations, capturing the mechanical behavior during the elastic stage is disclosed herein, which generally occurs when strain is within 10%. The compressive modulus is used to evaluate whether the architected structure can replicate the properties of adipose tissue. In one or more embodiments, the mechanical behavior of the structure is tailored by adjusting geometric parameters to achieve target stiffness and deformation characteristics associated with natural tissue.

In one or more embodiments, the plurality of architected structures is configured to provide conformability to the patient's body, such that the EBP 102 deforms under applied loads to conform to the contour of the chest wall. This conformability is achieved through selection of geometric parameters and material properties that enable localized deformation while maintaining overall structural integrity. In one or more embodiments, the conformability enhances comfort, improves contact with the skin, and reduces localized pressure points during use.

In one or more embodiments, the outer body of the EBP 102 is configured to provide aesthetic characteristics that approximate natural breast appearance, including surface texture, coloration, and contour. For example, the outer surface is formed or coated to match a patient's skin tone and texture and includes surface features that enhance realism. In one or more embodiments, aesthetic customization is selected based on patient preferences or derived from imaging data.

The range of the relative density of the architected structure is determined by matching the density of the designed structure to the target density. To further refine the relative density of the architected structure, the compressive modulus of the architected structure should also approximate the target compressive modulus of adipose tissue. Many studies have characterized the mechanical properties of both glandular and adipose tissues through compression testing conducted in both ex vivo and in vivo settings. In one or more embodiments, the relative density and the compressive modulus are jointly selected to satisfy one or more design objectives, including comfort, load balancing, and biomechanical compatibility.

For example, a target modulus is selected where the samples were taken in vivo from both healthy and cancerous tissues to prevent changes in modulus caused by the tissue sample extraction process. The moduli of the glandular tissue and the adipose tissue are about 7.5 kPa and about 3.3 kPa, respectively. Assuming the breast tissue consists solely of glandular and adipose tissues, the upper and lower bounds of the target modulus for the plurality of architected structures incorporating glandular tissue can be calculated using the Voigt and Reuss models, as outlined in Formula (IV).

$$\frac{{E}_{g} {E}_{a}}{{V}_{g} {E}_{a} + {V}_{a} {E}_{g}} \leq {E}_{t} \leq {V}_{g} {E}_{g} + {V}_{a} {E}_{a} \qquad ( IV )$$

Where, Et is the target modulus. V_{g} and Vₐ represent the volume of the glandular tissue (24%) and the adipose tissue (76%). As a result, the target modulus of the architected structure with glandular ranges from about 3.81 kPa to about 4.31 kPa. In one or more embodiments, the target modulus ranges are adjusted based on patient-specific anatomical data or desired performance characteristics.

Due to the highly nonlinear behavior of the plurality of architected structures, linear regression analysis is typically used to approximate the relationship between relative density and compressive modulus. The Gibson-Ashby model is the most widely recognized framework for analyzing the impact of relative density on the mechanical properties of cellular materials and has become the standard approach, as shown in Formula (V).

$$\frac{E}{{E}_{s}} = {C ( \frac{ρ}{{ρ}_{s}} )}^{n} \qquad ( V )$$

Where, E is the compressive modulus of the plurality of architected structures, while Es is that of the solid base material. C and n are the model parameters that can be determined by fitting existing or measured data. Usually, C ranges from about 0.1 to about 4.0, and n is generally set to about 2.0 for structures dominated by bending deformation and about 1 for those dominated by shearing deformation.

To determine the model parameters, experimental studies on the plurality of architected structures (e.g., gyroid structures) have been conducted which focus on the compressive modulus in the elastic stage. The normalized compressive modulus is defined as the ratio of the compressive modulus of the gyroid structures (E) to that of the compressive modulus solid printing material (Eₛ).

Using the Gibson-Ashby model, these data were fitted to derive a formula describing the effect of relative density on compressive modulus, as shown in Formula (V). The model parameters C and n may be determined for a selected architected structure, and the resulting relationship may be used to select a relative density that provides an effective compressive modulus corresponding to the tissue properties to be matched.

In one or more embodiments, the base material is selected from one or more printable soft materials, and the architected structure is designed to provide effective properties that approximate one or more target properties of breast tissue. The target properties may include an effective density, an effective Young's modulus, thermal conductivity, mechanical compliance, structural stability, or combinations thereof. In one or more embodiments, the target effective density is selected to approximate breast tissue and may be from about 0.4 g/cm³ to about 1.1 g/cm³. In one or more embodiments, the target effective Young's modulus is selected to approximate breast tissue and may be from about 0.3 kPa to about 50 kPa.

The subject matter disclosed above successfully mimicked the density and compressive modulus of breast tissue using a architected materials composite. By selecting geometric parameters such as relative density and wavelength, the architected structure is configured to approximate the density and compressive modulus of breast tissue.

### Thermal Conductivity

Thermal conductivity is another design factor, as many patients experience heat-related discomfort following a mastectomy. By incorporating structures configured for effective thermal regulation, the design can help dissipate excess heat, minimizing the likelihood of overheating and enhancing patient comfort. Ensuring thermal conductivity in the structure reduces heat buildup and promotes a cooler, more comfortable experience for the patient, addressing a common concern in post-operative care. In one or more embodiments, thermal conductivity is selected or tuned based on one or more design objectives, including user comfort, environmental conditions, and anticipated usage scenarios.

In one or more embodiments, the plurality of architected structures are further configured to promote airflow and ventilation through interconnected pore networks defined by the internal geometry. The airflow enhances convective heat transfer and facilitates dissipation of heat and moisture from the interface between the EBP 102 and the patient's skin. In one or more embodiments, the internal geometry is selected to increase surface area and permeability, thereby improving breathability and reducing moisture accumulation during extended wear.

Since solid lobe structures simulate glandular tissues, embodiments described herein contemplate the thermal conductivity of the architected structure with varied structural characteristics. With the relative density already determined to match the target density and compressive modulus, geometric configurations can be modulated by varying geometric parameters. Increasing certain geometric parameters, such as porosity wavelength, enhances the thermal conductivity of the plurality of architected structures. To establish this relationship, simulations were conducted to find the optimal geometric features that enable maximum thermal conductivity.

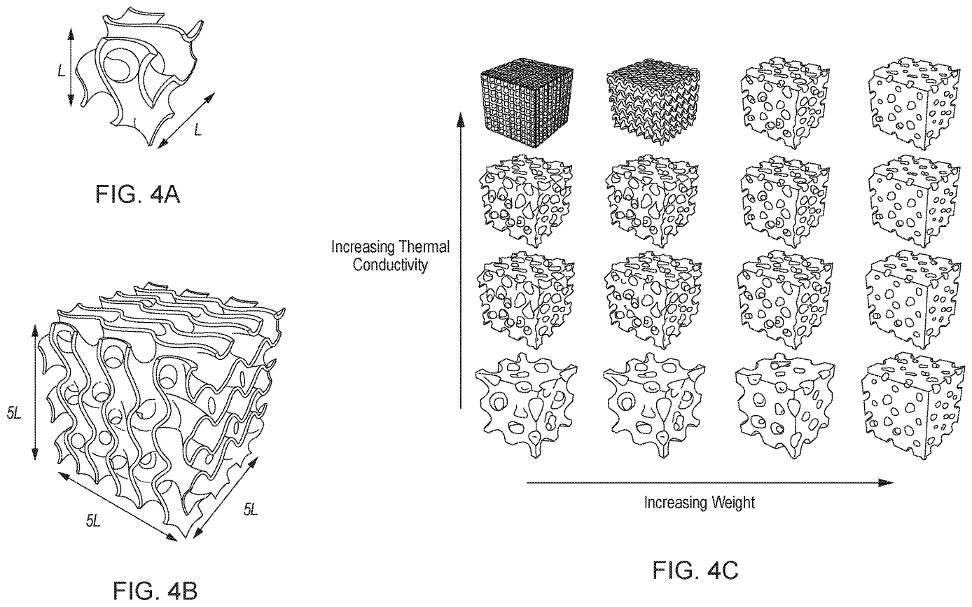

In particular, FIGS. 4A, 4B, and 4C illustrate example configurations of the plurality of architected structures corresponding to different selections of geometric parameters. FIGS. 4A, 4B, and 4C illustrate example variations in structural configuration and corresponding effects on mechanical and thermal performance. In one or more embodiments, these figures demonstrate how changes in geometric parameters, such as wavelength, influence both heat transfer characteristics and mechanical response of the structure.

The wavelength range was chosen to be sufficiently broad to identify the maximum thermal conductivity while remaining as close as possible to the target modulus. All the cubic samples have a side length of about 50 mm and a relative density close to about 28%. The normalized thermal conductivity A/As is defined as the ratio of thermal conductivity between an architected structure cubic (A) and the solid cubic (As). In one or more embodiments, normalized thermal conductivity is used as a design metric for comparing performance across different geometries and material configurations.

In one or more embodiments, a plurality of candidate architected material geometries are evaluated using simulation to determine effective density, stiffness, and thermal conductivity. The simulation results may be used to compare the candidate architected material geometries with one or more target properties associated with the tissue to be mimicked. Based on the comparison, an optimal or preferred architected material geometry may be selected to provide a desired balance among density matching, stiffness matching, and thermal conductivity matching. In one or more embodiments, the selected architected material geometry is identified as the geometry that minimizes a difference between the simulated effective properties and the target properties, subject to one or more constraints including manufacturability, structural stability, durability, or user comfort.

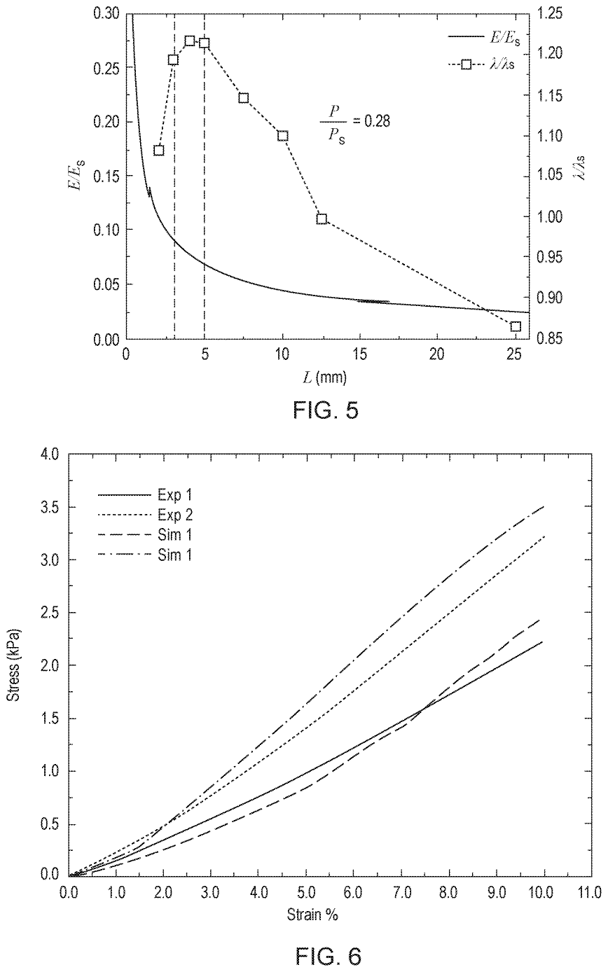

Example simulation results are shown in FIG. 5, which illustrates relationships between a geometric parameter (e.g., wavelength) and normalized mechanical and thermal properties of the architected structure. FIG. 5 illustrates that as the wavelength increases, the thermal conductivity of the plurality of architected structures initially increases before beginning to decrease. This trend can be attributed to structures with smaller wavelengths enhancing thermal convection and radiation due to their larger specific surface area. Conversely, structures with larger wavelengths facilitate solid heat transfer, as their heat transfer pathways are more direct and contain fewer cavities. FIG. 5 further illustrates that different values of the geometric parameter correspond to different combinations of mechanical and thermal performance. A range of values of the geometric parameter are identified to provide desirable thermal conductivity while maintaining acceptable mechanical properties. For example, based on this analysis, gyroid structures with wavelengths between 3.0 mm and 5.0 mm achieve higher thermal conductivity at a relative density of 0.28. Considering the rapid decrease in normalized compressive modulus with increasing wavelength within this range, a wavelength of 5.0 mm was selected as a design demonstration for further experimental testing. In one or more embodiments, the relative density of the plurality of architected structures are selected within a range of about 0.20 to about 0.50, depending on desired mechanical and thermal performance characteristics. The relative density range depends on the chosen printing materials that match the breast tissues. Although the resulting structure may not exactly match all target properties, the selected configuration provides a desired balance of performance characteristics suitable for the intended application. It is noted that FIG. 5 shows one example of simulation results for one architected structure, and if a different material or different geometric parameters are used, the simulation results adjust accordingly.

### Results

Numerical simulations were conducted to evaluate the mechanical properties and thermal conductivity of the plurality of architected structures with and without embedded glandular cells, aiming to replicate the properties of breast tissue. Subsequently, 3D-printed samples were fabricated and experimental tests were performed to assess their mechanical performance. In one or more embodiments, the plurality of architected structures correspond to example configurations such as Type 1 refers to samples composed solely of the architected structure to mimic adipose tissues, while Type 2 includes both the architected structure and simulated glandular cells to reproduce the overall breast tissues. Examples of the Type 1 and Type 2 structures are shown in FIG. 9. The relative density of the Type 2 sample was measured at 0.444, showing a minimal deviation of 1.97% from the designed density of 0.453. In one or more embodiments, the results demonstrate the ability to fabricate structures with controlled geometric parameters that closely match target design values.

The compressive behaviors of both samples is evaluated through finite element analysis and experimental testing. FIG. 6 illustrates the stress-strain curves of the plurality of architected structures obtained from both simulation and experimental testing, where stress is defined as the force per unit cross-sectional area and strain is the ratio of displacement to the original length of the structure in the loading direction. FIG. 6 illustrates the relationship between applied compressive load and resulting deformation for different structure configurations, including structures with and without simulated Type 2 glandular regions. The comparison between simulation and experimental curves demonstrates the extent to which computational models predict mechanical behavior of the fabricated structures.

Both samples remained within the elastic regime, and the compressive moduli of the samples was calculated at a strain level of about 2.0%. Experimental testing showed compressive moduli for the Type 1 and Type 2 samples are 17.71 kPa and 24.40 kPa, with normalized moduli of 0.059 kPa and 0.081 kPa, respectively. In comparison, simulations yielded compressive moduli of 14.47 kPa and 22.58 kPa for the two samples, with normalized values of 0.048 kPa and 0.075 kPa, respectively. For the architected structure without glandular cells, the analytical normalized modulus, calculated using Formula (V), is 0.066 kPa. Additionally, using the Voigt and Reuss models in Formula (IV), the analytical normalized modulus for the architected structure with glandular cells was estimated to range from 0.085 to 0.290. Table 1 summarizes the normalized compressive moduli of both samples derived from analytical, numerical, and experimental analyses. These results indicate that the analytical predictions align with experimental values within about 5% to about 11% margin, while the simulation results show approximately a 10% lower modulus compared to the experimental data. In one or more embodiments, the agreement between analytical, numerical, and experimental results validates the use of the described design framework for predicting mechanical behavior based on selected geometric parameters.

TABLE 1
Normalized compressive moduli obtained using different methods

| | Analytical result | Simulation | Experiment |
|---|---|---|---|
| Type 1 | 0.066 | 0.048 | 0.059 |
| Type 2 | 0.085-0.290 | 0.075 | 0.081 |

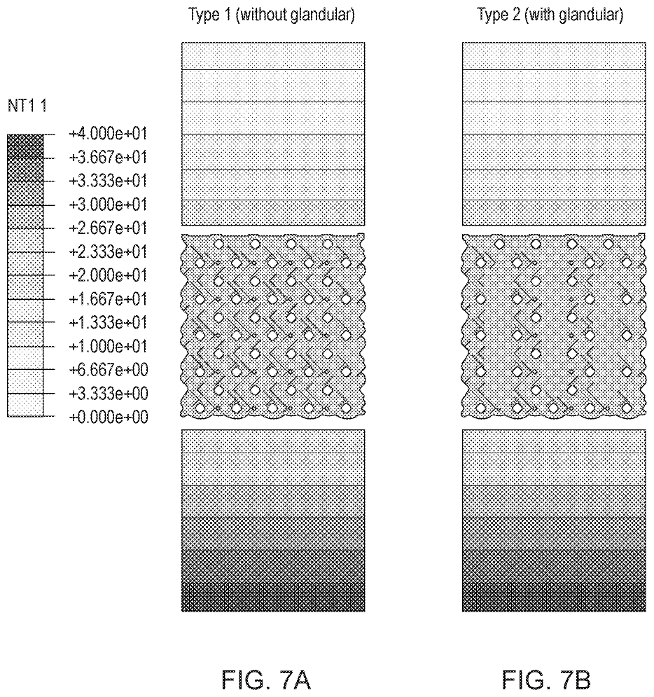

Thermal conductivity analysis was conducted on the plurality of architected structures to simulate their heat transfer behavior, accounting for solid conduction, radiation, and convection. The normalized thermal conductivity (N/As) was found to be 1.215 for the plurality of architected structures mimicking adipose tissue and 1.094 for those incorporating both adipose and glandular tissues. FIG. 7 illustrates example temperature distributions within the plurality of architected structures under thermal loading conditions. FIG. 7 depicts spatial variation in temperature across the structure, illustrating heat transfer pathways and the influence of internal geometry on thermal behavior.

For example, steady-state temperature distributions indicate that the presence of glandular tissue reduces the thermal conductivity of the architected structure by approximately 10%. The architected structure containing glandular tissue required about 270 seconds to reach a steady state, compared to 240 seconds for the structure without glandular tissue. FIG. 7 demonstrates that inclusion of the glandular regions alters hear transfer characteristics by modifying internal conduction pathways and thermal resistance. Despite this reduction, the simulation results show an overall increase in thermal conductivity of 21.5% and 9.4%, respectively, compared to homogeneous structures commonly used in prosthetic applications. In one or more embodiments, the results demonstrate that the plurality of architected structures are configured to enhance thermal performance relative to conventional homogeneous prostheses, while maintaining desired structural and mechanical characteristics.

### Methods and Materials

### Thermal Conductivity Analysis

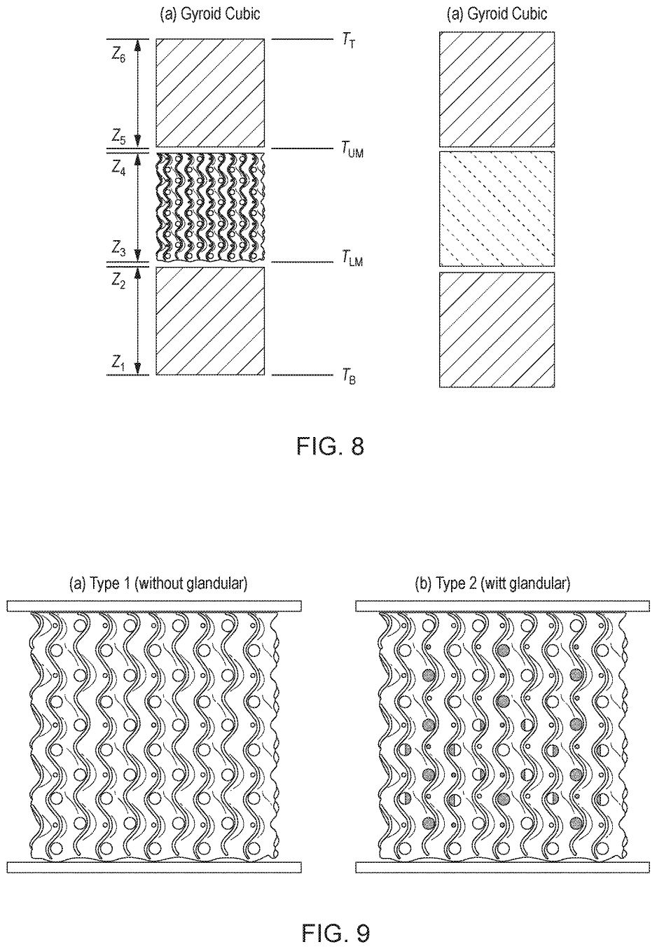

The thermal conductivity of the plurality of architected structures was determined using the comparative longitudinal heat flow technique. Finite element models were developed to simulate the test setup, placing the sample between two stainless steel reference blocks (thermal conductivity, λ=16.2 W·m⁻¹·K⁻¹ at 293 K), as shown in FIG. 8. In FIG. 8, Z represents the height of the blocks, and T denotes the temperature at various positions. A 4-node linear heat transfer tetrahedron element (DC3D4) with an approximate mesh size of 0.5 mm was employed for the lattice structures, while an 8-node linear heat transfer brick element was used for the two reference blocks.

Considering that the thermal conductivity of biological tissues typically ranges from about 0.4 W·m⁻¹·K⁻¹ to about 2.25 W·m⁻¹·K⁻¹, corresponding to the thermal conductivity range of biological tissue and providing improved thermal regulation relative to homogeneous prosthetic materials. The thermal conductivity depends on the chosen printing materials that match the breast tissues as well as patient input.

The thermal conductivity of the test sample was designed to be near 1 W·m⁻¹·K⁻¹. For efficient thermal contact during analysis, a silver sheet with high thermal conductivity (>=420 W·m⁻¹·K⁻¹) was placed between the test sample and each reference block to model a realistic boundary conditions. The bottom surface of the lower reference block was maintained at 310 K, approximating body temperature, while the top surface of the upper reference block was cooled to 273 K. The ambient temperature was set to 293 K. In addition to solid conduction, the effects of radiation and convection were incorporated into the analysis. The emissivity of the material was set at 0.3, and a film coefficient of 15 W·m⁻²·K⁻¹ was used to model heat exchange with the surrounding air.

Transient heat transfer analysis was conducted using finite element analysis. Steady-state conditions were considered achieved when the temperature drift remained below 0.1 K/min at the specified temperatures. After reaching a steady state, the thermal conductivity of the test sample was determined using Formula (VI).

$$λ = ( \frac{{λ}_{M}}{2} ) ( \frac{{Z}_{4} - {Z}_{3}}{{T}_{UM} - {T}_{LM}} ) ( \frac{{T}_{LM} - {T}_{B}}{{Z}_{2} - {Z}_{1}} + \frac{{T}_{T} - {T}_{UM}}{{Z}_{6} - {Z}_{5}} ) \qquad ( VI )$$

In Formula (VI), Z represents spatial position along a measurement axis, T represents temperature at corresponding positions, LM refers to a lower measurement region, UM refers to an upper measurement region, and B refers to a boundary or reference interface. A represents the thermal conductivity of the architected structure, and Am represents the thermal conductivity of the base material. In one or more embodiments, the simulations are used to evaluate the effect of geometric parameters on heat transfer behavior and to inform selection of design parameters for achieving target thermal performance.

### Mechanical Behavior Analysis

Finite element models of the plurality of architected structures with a wavelength of 5 mm were developed, as illustrated in FIG. 9. Tetrahedral elements (C3D4) with an approximate mesh size of 0.5 mm were employed. Since the analysis focused exclusively on the compressive modulus of the structure, only the elastic properties of the printing material were specified. The printing material, a soft polymer material, includes a density of 1.2 g/cm³, and the elastic modulus was set to 300 kPa. In one or more embodiments, the EBP 102 is fabricated from a material including an elastic modulus ranging from about 0.01 GPa to about 0.1 GPa. In one or more embodiments, other material having similar mechanical properties are used. In one or more embodiments, the compressive modulus of the plurality of architected structures is selected within a range of about 2 kPa to about 30 kPa to approximate the mechanical behavior of adipose and glandular tissues under expected loading conditions. The compressive modulus range depends on the chosen printing materials that match the breast tissues.

The sample was positioned between two rigid plates, with general contact defined between the plates and the structure. The lower plate was fixed, while the upper plate applied compression to the cube at a rate of 1 mm/min, up to a maximum displacement of 5 mm. The explicit dynamic finite element solver was utilized to capture the behavior of the plurality of architected structures under these conditions. The simulations are used to predict mechanical response of the structure and to evaluate the effect of geometric parameters on stiffness and deformation behavior.

### Printing Materials and Compressive Test Method

In embodiments described herein, a soft, deformable polymer material is selected as a base material due to its mechanical properties For example, the soft, deformable polymer material includes a Shore 00 hardness of approximately 27, and in one or more embodiments within a range of about 10 to about 50 depending on desired tactile and mechanical characteristics. Materials as disclosed are used in anatomical models for surgical training, as the disclosed materials offer consistent stiffness and tunable surface friction through various finishing treatments, as demonstrated in prior studies.

These properties make the disclosed materials well-suited for replicating the compliance of natural breast tissue. As an example, the samples were fabricated using a Stratasys J750 Digital Anatomy Printer, employing VeroPureWhite rods embedded within an Agilus30CLR matrix. However, it should be understood that any multi-material AM system may be used to fabricate the plurality of architected structures disclosed herein. The models were printed as single assemblies using a high-mix mode to ensure structural integrity and material consistency. Post-processing involved the removal of support material through a combination of manual scraping, water jetting, and an agitated alkaline bath.

The weights of the printed samples were measured using a precision balance, yielding values of 40.55 g and 66.61 g, respectively. The relative density of the architected structure sample without glandular features was 27.03%, closely aligning with the target relative density of 28%. Compression tests were performed using a universal testing machine at a 1 mm/min loading speed. The force and displacement data were recorded throughout the tests by the testing system. In one or more embodiments, the fabrication and testing processes are used to validate predicted properties of the plurality of architected structures and to refine design parameters for improved performance.

### Patient Care Method

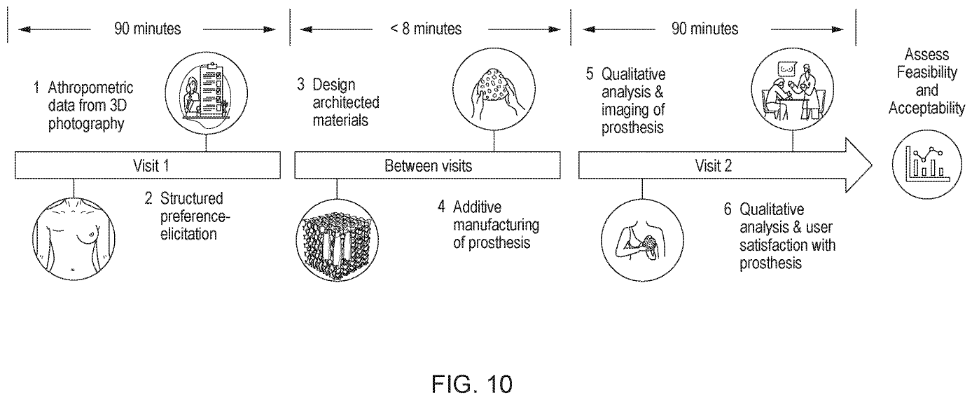

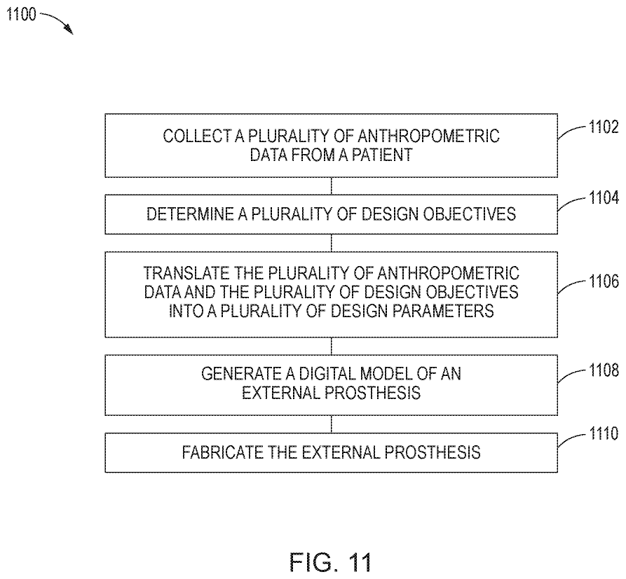

FIG. 10 illustrates the patient care method for providing an EBP 102 that is patient specific. FIG. 11 illustrates a flow diagram of a method 1100 for designing, manufacturing, and providing the EBP 102 for the patient 106, according to one or more embodiments. The method 1100 enables patient-specific customization of prosthetic devices by integrating anatomical data, design objectives, computational modeling, and AM techniques. In one or more embodiments, the method facilitates a comprehensive workflow in which patient-specific inputs are translated into design parameters that define both the external geometry and internal structure of the prosthesis. In one or more embodiments, the method 1100 further includes iterative refinement of the design based on simulation results, user feedback, or clinical input to achieve desired performance characteristics. In one or more embodiments, the method supports the creation of one or more EBPs tailored to different use scenarios, such as daily wear, physical activity, or special events, thereby providing a flexible and adaptive prosthetic solution that evolves with patient needs over time. For example, a daily-wear configuration may be designed to prioritize lower relative density, reduced stiffness, and moderate thermal conductivity to promote long-term comfort, breathability, and thermal management during extended use. A physical-activity configuration may target increased relative density, higher compressive stiffness, and improved thermal conductivity to accommodate elevated mechanical loading and increased metabolic heat generation during dynamic movement. A low-activity or special-event configuration may target the lowest relative density and stiffness values, optimizing comfort, aesthetics, and reduced weight for short-duration wear. In one or more embodiments, the relative density, compressive modulus, and thermal conductivity of each configuration differ by a clinically meaningful degree sufficient to produce distinct functional performance characteristics across the range of intended use scenarios. These target values may be further adjusted based on patient anatomy, body weight, activity level, and clinical objectives. At operation 1102, a plurality of anthropometric data is collected from the patient. The plurality of anthropometric data includes three-dimensional (3D) surface scans of the chest wall, measurements of breast geometry, body symmetry data, imaging data such as magnetic resonance imaging (MRI) or computed tomography (CT) scans, or combinations thereof. In one or more embodiments, the plurality of anthropometric data further includes the capture of a contralateral breast to establish symmetry and reference geometry. The plurality of anthropometric data that is collected characterizes anatomical features including volume, contour, and spatial distribution of tissue. Additionally, patient-specific factors such as age, body mass index (BMI), activity level, and sensitivity of post-surgical regions are also considered during operation 1102. In one or more embodiments, patient input regarding comfort, preferred fit, and intended use scenarios is also collected during operation 1102. The plurality of anthropometric data provides a foundation for generating the EBP 102 tailored to the individual anatomy and physiological needs of the patient.

At operation 1104, a plurality of design objectives are determined. The design objectives include target density, mechanical compliance, thermal conductivity, weight distribution, and aesthetic appearance. In one or more embodiments, the design objectives are defined based on patient preferences, clinician input, and/or intended use scenarios. For example, a patient may prioritize lightweight construction for daily wear, increased thermal dissipation for warm environments, or enhanced structural support for physical activity. The design objectives also account for biomechanical considerations such as load balancing and postural alignment. For example, distribution of weight across the chest wall to reduce asymmetric loading, minimize strain on the shoulders and back, and improve overall balance during standing, walking, or other activities. In one or more embodiments, the EBP 102 is configured to approximate the mass distribution and center of gravity of natural breast tissue, thereby reducing compensatory posture adjustments and associated discomfort. In one or more embodiments, the biomechanical considerations are particularly relevant for unilateral prostheses, where imbalance between sides of the body may otherwise lead to fatigue or musculoskeletal strain over time. In one or more embodiments, the design objectives include selection of target ranges for density, mechanical compliance, and thermal conductivity that vary based on intended use scenarios, such as lower density and higher thermal conductivity for active use conditions and higher structural support for daily wear conditions. In one or more embodiments, different use scenarios correspond to different target ranges for one or more design parameters. For example, the EBP 102 configured for physical activity includes a relative density in a lower portion of a defined range and a higher thermal conductivity, while the EBP 102 configured for everyday wear includes a higher relative density and increased structural support.

At operation 1106, the plurality of anthropometric data and the plurality of design objectives are translated into a plurality of design parameters. The design parameters include geometric parameters associated with the architected structure, such as relative density, wavelength, unit cell size, wall thickness, topology, and spatial distribution of structural regions. In one or more embodiments, computational models, analytical relationships, and/or numerical simulations are used to determine relationships between the design parameters and resulting physical properties, including mechanical and thermal behavior. In one or more embodiments, translation of the plurality of anthropometric data and design objectives include mapping anatomical regions to corresponding structural regions of the prosthesis, including assignment of different parameter values to simulate heterogeneous tissue composition. Operation 1106 includes selecting parameter values that satisfy one or more design objectives or optimizing the parameters based on multi-objective criteria. In one or more embodiments, the design parameters are iteratively refined based on simulation outputs or predicted performance characteristics.

At operation 1108, a digital model of an external prosthesis is generated using the plurality of design parameters. In one or more embodiments, the external prosthesis is the EBP 102. The digital model includes the plurality of architected structures, such as gyroid-based or lattice-based geometries, arranged to form an internal structure of the prosthesis. In one or more embodiments, the digital model incorporates multiple regions, including relatively porous regions configured to simulate adipose tissue and relatively solid or less porous regions configured to simulate glandular tissue. The model is generated using parametric design techniques in which one or more geometric parameters are varied to produce candidate designs. In one or more embodiments, a plurality of candidate digital models are generated and evaluated. In one or more embodiments, the digital model is iteratively refined based on simulation results to better achieve target performance characteristics. In one or more embodiments, the digital model further includes an outer surface geometry configured to match the patient anatomy and an internal structure configured to achieve desired functional properties.

In one or more embodiments, the method 1100 further includes receiving feedback from the patient after initial use of the EBP 102 and updating one or more design parameters based on the feedback. The feedback relates to comfort, fit, thermal performance, weight distribution, or aesthetic preferences. The updated design parameters are used to generate a revised digital model and fabricate an updated prosthesis, thereby enabling an iterative, patient-centered design process.

In one or more embodiments, the EBP 102 is fabricated using AM techniques to form the internal structure and external geometry defined by the digital model. For example, 3D printing methods are used. For example, material jetting, stereolithography (SLA), digital light processing (DLP), fused deposition modeling (FDM), selective laser sintering (SLS), other layer-by-layer fabrication processes, or combinations thereof are used to fabricate the EBP 102. In one or more embodiments, multi-material AM systems are used to fabricate different regions of the EBP 102 with distinct material properties and/or geometric parameters, including relatively porous regions configured to simulate adipose tissue and relatively solid regions configured to simulate glandular tissue. In one or more embodiments, the AM process is selected to achieve a desired level of resolution and geometric fidelity, enabling accurate reproduction of the plurality of architected structures defined in the digital model. High-resolution fabrication is used to ensure consistency of unit cell geometry, repeatability across builds, and reliable control of mechanical and thermal properties.

In one or more embodiments, the EBP 102 is formed from soft, deformable polymer materials configured to approximate the mechanical response of biological tissue, including elastomeric materials, photopolymers, silicones, or combinations thereof. For example, materials having mechanical properties similar to human tissue are used to achieve desired compliance and tactile characteristics. In one or more embodiments, the fabrication process includes printing the EBP 102 as a unitary structure or as multiple components that are subsequently coupled. Post-processing operations, such as removal of support material, cleaning, curing, or surface finishing, are performed to prepare the EBP 102 for use.

At operation 1110, the EBP 102 is secured to the patient using one or more attachment mechanisms (e.g., the EBP support structure 104) configured to maintain position, comfort, and stability during use. The attachment mechanisms include, for example, medical-grade adhesives, adhesive-backed interfaces, garments (e.g., bras or compression garments), straps, or combinations thereof. In one or more embodiments, the outer surface of the EBP 102 is configured to interface with the patient's skin, including through conformal contact with the chest wall to enhance retention and comfort. The internal structure of the EBP 102, including the architected structure, contributes to improved stability by reducing weight and enabling better distribution of forces across the contact area.

In one or more embodiments, the EBP 102 is adapted over time to accommodate changes in patient anatomy, such as those occurring during post-surgical healing, swelling reduction, or long-term physiological changes. The method 1100 includes generating an updated EBP 102 at different time points based on updated anthropometric data or patient feedback.

In one or more embodiments, the method 1100 further includes selecting or customizing the attachment mechanism based on patient-specific factors, including skin sensitivity, activity level, and intended use scenario. For example, a first attachment configuration is selected for everyday wear to prioritize comfort and ease of removal, while a second attachment configuration is selected for physical activity to provide increased stability and retention. In one or more embodiments, the attachment mechanism is integrated with or coordinated with the design of the EBP 102 to provide a secure and comfortable fit across a range of use conditions.

In one or more embodiments, the EBP 102 is configured to facilitate cleaning and maintenance for repeated use. In one or more embodiments, the EBP 102 includes an outer covering or interface layer that is removable from the internal structure to enable washing or replacement. In one or more embodiments, the removable covering is formed from a textile, polymer film, or other flexible material configured to contact the patient's skin, thereby providing a hygienic and comfortable interface. The covering is removably coupled to the EBP 102 using, for example, mechanical fasteners, friction fit, elastic retention, or other attachment mechanisms.

In one or more embodiments, the removable covering is washable using standard laundering processes, allowing the patient to maintain cleanliness of the EBP 102 during regular use. In one or more embodiments, the internal structure of the EBP 102 is configured to resist moisture accumulation and facilitate drying, including through the use of the plurality of architected structures that promote airflow and reduce fluid retention. In one or more embodiments, multiple coverings are provided for a single EBP 102, enabling the patient to interchange coverings based on hygiene needs, comfort preferences, or aesthetic considerations. This approach enhances usability and supports long-term use of the EBP 102 in a variety of daily and activity-specific scenarios.

In one or more embodiments, the internal structure of the EBP 102 is modular, such that one or more internal components or regions are selectively removed, replaced, or reconfigured. For example, an architected structure insert having different geometric parameters are interchangeably used to modify mechanical or thermal performance of the prosthesis. The modularity enables further customization and adaptation of the EBP 102 over time without requiring fabrication of an entirely new device.

Overall, embodiments of the present disclosure provide EBPs and associated methods for designing and manufacturing EBPs that incorporate the plurality of architected structures to achieve desired physical and functional properties. By leveraging the plurality of anthropometric data that is patient-specific, defined design objectives, and computational modeling, the disclosed approach enables translation of geometric parameters into controlled mechanical, thermal, and density characteristics of the prosthesis. The resulting EBPs include the plurality of architected structures that are internal, such as gyroid-based or other lattice-based geometries, configured to simulate the heterogeneous composition of natural breast tissue. Through AM techniques, the disclosed method enables fabrication of customized prostheses having tailored performance characteristics, including improved weight distribution, enhanced thermal regulation, and biomechanical compatibility.

The disclosed systems and methods provide advantages over conventional prosthetic devices by enabling patient-specific customization not only of external shape but also of internal structure and function. This approach may improve comfort, reduce heat accumulation, enhance postural balance, and provide a more natural feel and appearance. Additionally, the ability to generate multiple prostheses optimized for different use scenarios, such as daily wear, physical activity, or specialized applications, provides increased flexibility and improved quality of life for patients. Accordingly, the present disclosure provides a versatile and scalable framework for the design and manufacture of advanced prosthetic devices that address longstanding challenges in the field.

While the foregoing is directed to embodiments of the present disclosure, other and further embodiments of the disclosure may be devised without departing from the basic scope thereof, and the scope thereof is determined by the claims that follow.

What is claimed is:

1. An external prosthesis comprising:
an outer body comprising a shape configured to approximate a breast; and
an internal structure disposed within the outer body, the internal structure comprising a plurality of architected structures having an internal geometry, the internal geometry comprising a plurality of unit cells arranged to define a three-dimensional topology, the plurality of unit cells comprising:
a defined density;
a mechanical compliance; and
a thermal conductivity.

2. The external prosthesis of claim 1, further comprising:
a first region comprising a first architected structure, wherein the first architected structure is configured to simulate an adipose tissue; and
a second region comprising a second architected structure, wherein the second architected structure is configured to simulate a glandular tissue, wherein the first region and the second region are integrated to form a unitary structure.

3. The external prosthesis of claim 1, wherein the internal geometry is triply periodic minimal surface (TPMS)-based, spinodal, lattice-based, stochastic cellular, or combinations thereof.

4. The external prosthesis of claim 1, further comprising a removable outer covering configured to interface with a patient's skin.

5. The external prosthesis of claim 1, wherein the plurality of architected structures have a relative density in a range of about 0.20 to about 0.50.

6. The external prosthesis of claim 1, wherein the plurality of architected structures comprises a compressive modulus in a range of about 2 kPa and about 30 kPa.

7. The external prosthesis of claim 1, wherein the plurality of architected structures comprise the thermal conductivity is about 0.4 W·m⁻¹·K⁻¹to about 2.25 W·m⁻¹·K⁻¹.

8. An external breast prosthesis (EBP) comprising:
a first region comprising a first architected structure, wherein the first architected structure is configured to simulate an adipose tissue; and
a second region comprising a second architected structure, wherein the second architected structure is configured to simulate a glandular tissue, wherein the first region and the second region are integrated to form a unitary structure.

9. The EBP of claim 8, wherein the first architected structure and the second architected structure comprise an internal geometry, the internal geometry comprising a plurality of unit cells arranged to define a three-dimensional topology, the plurality of unit cells comprising:
a defined density;
a mechanical compliance; and
a thermal conductivity.

10. The EBP of claim 8, wherein the first architected structure and the second architected structure comprise a gyroid structure, a triply periodic minimal surface (TPMS) structure, or combinations thereof.

11. The EBP of claim 8, further comprising a removable outer covering configured to interface with a patient's skin.

12. The EBP of claim 9, wherein the first architected structure and the second architected structure have a relative density in a range of about 0.20 to about 0.50.

13. The EBP of claim 9, wherein the first architected structure and the second architected structure comprise a compressive modulus in a range of about 2 kPa to about 30 kPa.

14. The EBP of claim 9, wherein the first architected structure and the second architected structure comprise the thermal conductivity is about 0.4 W·m⁻¹·K⁻¹ to about 2.25 W·m⁻¹·K⁻¹.

15. A method of forming an external breast prosthesis (EBP), comprising:
collecting a plurality of anthropometric data from a patient;
determining a plurality of design objectives based on at least the anthropometric data;
translating the plurality of anthropometric data and the plurality of design objectives into a plurality of design parameters associated with a plurality of architected structures, the design parameters comprising relative density, wavelength, unit cell geometry, topology, or combinations thereof;
generating a digital model of the EBP based on the plurality of design parameters, the digital model including an internal structure comprising the plurality of architected structures; and
forming the EBP based on the digital model using an additive manufacturing process.

16. The method of claim 15, wherein collecting the plurality of anthropometric data comprises obtaining at least one of a three-dimensional scan, magnetic resonance imaging (MRI) data, or computed tomography (CT) data of the patient.

17. The method of claim 15, wherein the plurality of design objectives comprises one or more of target density, mechanical compliance, thermal conductivity, weight distribution, or aesthetic profile.

18. The method of claim 15, wherein translating the plurality of anthropometric data and the plurality of design objectives comprises mapping anatomical regions of the patient to corresponding regions of the EBP, including assigning different design parameters to simulate adipose tissue and glandular tissue.

19. The method of claim 15, wherein forming the EBP comprises using at least one of material jetting, stereolithography (SLA), digital light processing (DLP), fused deposition modeling (FDM), or selective laser sintering (SLS).

20. The method of claim 15, further comprising forming a plurality of EBPs for the patient, each EBP being configured with different design parameters corresponding to different use scenarios.
